(19) United States
(12) Patent Application Publication
(10) Pub. No.: US 2024/0216375 A1
(43) Pub. Date: Jul. 4, 2024

(54) USE OF SYMPATHETIC ACTIVATION INHIBITOR AND/OR ALPHA 1-ADRENERGIC RECEPTOR INHIBITOR IN PREPARING MEDICINE FOR TREATING DRY EYES

(71) Applicant: Eye Institute of Shandong First Medical University (Shandong Eye Institute, Qingdao Eye Hospital of, Qingdao (CN)

(72) Inventors: Lixin Xie, Qingdao (CN); Qingjun Zhou, Qingdao (CN); Mingli Qu, Qingdao (CN); Sai Zhang, Qingdao (CN)

(21) Appl. No.: 17/924,198

(22) PCT Filed: Aug. 18, 2022
(86) PCT No.: PCT/CN2022/113244

§ 371 Date: Nov. 9, 2022

(30) Foreign Application Priority Data
May 9, 2022 (CN) 202210497086.8

(51) Int. Cl. A61K 31/517; A61K 31/18; A61K 31/4045; A61K 45/00; A61P 27/02

(52) U.S. Cl. CPC A61K 31/517; A61K 31/18; A61K 31/4045; A61K 45/00; A61P 27/02

(57) ABSTRACT

A sympathetic activation inhibitor and/or an α1-adrenergic receptor inhibitor in preparing a medicine for treating dry eyes and belonging to the technical field of medicines for treating dry eyes, and a use of a sympathetic activation inhibitor and/or an α1-adrenergic receptor inhibitor in preparing a medicine for treating or relieving dry eye syndrome. The medicine prepared by the use can antagonize an α1-adrenergic receptor (including an α1a-adrenergic receptor) or inhibit sympathetic activation, and can relieve and treat dry eye symptoms.

### TECHNICAL FIELD

The present disclosure relates to the technical field of medicines for treating dry eyes and particularly relates to use of a sympathetic activation inhibitor and/or an α1-adrenergic receptor inhibitor in preparing a medicine for treating dry eyes.

### BACKGROUND

Dry eye syndrome, also known as keratoconjunctivitis sicca, dry eye, dysfunctional tear syndrome, etc., is a public health problem affecting the life quality of hundreds of millions of people worldwide. The dry eye is characterized by tear film instability and ocular surface damage due to tear deficiency or excessive evaporation, accompanied with various maladaptive symptoms of the eyes, and is a multifactorial induced tear and ocular surface disease. The dry eye is characterized by reduced tear volume, dry eye surface, fatigue and discomfort, and foreign body sensation, and serious syndromes of damage to the epithelium of the eye surface and impaired vision. Tear secretion is mainly regulated by keratoconjunctival sensory afferents and lacrimal sympathetic and parasympathetic efferents. Traditionally, it is considered that parasympathetic nerves are a main pathway for regulating tear secretion of the lacrimal gland by secreting acetylcholine (ACh) to bind to muscarinic acetylcholine receptor M3 (M3AChR) of acinar cells to induce secretion of water, proteins and electrolytes. The mechanism of sympathetic nerves in regulating and controlling the tear is unclear.

Classical dry eye pathogenesis includes tear film instability, high tear osmolarity, ocular surface inflammation and damage, neurosensory abnormalities, and the like. Currently, the dry eye is mainly treated by eye drops including artificial tears, anti-inflammatory eye drops (such as cyclosporine, rapamycin and the like), eye drops promoting secretion of mucoprotein (such as diquafosol) and the like. The eye drops target each layer of the ocular surface. Some medicines can relieve the dry eye aiming at multiple targets at the same time, for example, the diquafosol can improve functions of a tear film lipid layer and promote secretion of water and mucoprotein at the same time. These methods all mainly relieve local symptoms, treat symptoms rather than root causes, and thus have certain limitations. At present, there is no medicine for targeting the lacrimal gland and promoting tear secretion in the market. Therefore, finding a novel high-efficiency, targeting and cheap method for promoting tear secretion is a gospel for patients and has wide and positive influence and significance in clinical application.

### SUMMARY

The present disclosure aims to provide use of a sympathetic activation inhibitor and/or an α1-adrenergic receptor inhibitor in preparing a medicine for treating dry eyes. The medicine prepared by the use of the present disclosure can antagonize an α1-adrenergic receptor (including an α1a-adrenergic receptor) or inhibit sympathetic activation, and can relieve and treat dry eye symptoms.

The present disclosure provides use of a sympathetic activation inhibitor and/or an α1-adrenergic receptor inhibitor in preparing a medicine for treating or relieving dry eye syndrome.

Preferably, the dry eye syndrome may include aqueous-deficient dry eye syndrome or diabetes-associated dry eye syndrome.

The present disclosure further provides use of a sympathetic activation inhibitor and/or an α1-adrenergic receptor inhibitor in preparing a medicine for increasing tear secretion and/or reduce inflammation of a lacrimal gland and an ocular surface.

Preferably, the α1-adrenergic receptor inhibitor may include one or more of prazosin, terazosin, doxazosin, alfuzosin, and trimazosin.

Preferably, the α1-adrenergic receptor inhibitor may include an α1a-adrenergic receptor inhibitor.

Preferably, the α1a-adrenergic receptor inhibitor may include tamsulosin and/or silodosin.

Preferably, the sympathetic activation inhibitor may include a medicine for chemical sympathectomy, a norepinephrine inhibitor or a sympathetic ganglion blocking medicine; the medicine for chemical sympathectomy may include 6-hydroxydopamine; the norepinephrine inhibitor may include one or more of bretylium, reserpine, and guanethidine; and the sympathetic ganglion blocking medicine may include trimetaphan camsilate and/or mecamylamine.

Preferably, the α1-adrenergic receptor inhibitor or an agent inhibiting sympathetic activation may have a concentration of 1-100 mg/kg in the medicine.

Preferably, a dosage of the medicine may include an oral medicine, an injection, a sustained release tablet, a nasal spray or an embedded sustained release tablet.

The present disclosure provides use of a sympathetic activation inhibitor and/or an α1-adrenergic receptor inhibitor in preparing a medicine for treating dry eyes. The medicine prepared by the use of the present disclosure can antagonize an α1-adrenergic receptor (including an α1a-adrenergic receptor) or inhibit sympathetic activation, and can relieve and treat dry eye symptoms, increase tear secretion, and reduce inflammation of a lacrimal gland and an ocular surface. A test result shows dense sympathetic innervations in lacrimal gland for the first time through a clearing and staining technique by the present disclosure, suggesting that the lacrimal gland has a physiological basis for sympathetic nerves regulating. An increase of a norepinephrine (NE) level was detected both in a scopolamine-induced dry eye animal model and a diabetes-associated dry eye model, indicating that occurrence of dry eye in mice is accompanied by overactivation of sympathetic nerves. Sympathetic neurotoxin, 6-hydroxydopamine (6-OHDA), is used to specifically clear sympathetic nerves in dry eye model mice. It is found that the tear secretion of the 6-OHDA-intervened mice is significantly increased and the dry eye symptoms are alleviated, indicating that inhibition of sympathetic activation can effectively reduce severity of dry eye. Sympathetic nerves act mainly by releasing NE to bind to an adrenergic receptor on a target cell. Through quantitative PCR detection in the present disclosure, it is found that a lacrimal gland cell expresses four receptor types, α1a, α1d, β1, and β2, wherein the expression level of the α1a receptor is the highest, indicating that the α1a receptor is a key target receptor for sympathetic nerves to regulate tear secretion of a lacrimal gland. In the present disclosure, α1d and β receptor inhibitors are used as a control, and an intervention effect of the α1a and α1 receptor inhibitors on dry eye is evaluated. It is found that compared with untreated dry eye mice, the α1a and α1 receptor inhibitors can significantly increase tear secretion and reduce severity of dry eye, while the α1d and the β receptor inhibitors have no significant intervention effect. The α1a adrenergic receptor inhibitor is a class of medicines capable of selectively binding to an α1a adrenergic receptor, thereby block the combination of a neurotransmitter and catecholamine with the α1a receptor. The al adrenergic receptor inhibitor has an effect on inhibiting α1a, α1b, and α1d receptors at the same time.

The present disclosure provides a new theoretical basis for treating dry eye, targets sympathetic nerves and/or an α1a adrenergic receptor, and provides α1a- and α1-adrenergic receptor inhibitors or sympathetic activation inhibitors and use of other intervention means in targeting sympathetic nerves in preparing a medicine for treating dry eye and diabetes-associated dry eye.

### BRIEF DESCRIPTION OF THE DRAWINGS

FIG. 1 shows tear secretion in scopolamine treated dry eye model mice provided by example 1 of the present disclosure;

FIG. 2 shows corneal staining by fluorescein sodium 7 days after treated with scopolamine provided by the present disclosure;

FIG. 3 shows expression levels of inflammatory factors of the lacrimal glands (left) and the corneas (right) 7 days after treated with scopolamine provided by the present disclosure;

FIG. 4 shows a change of a norepinephrine (NE) level in blood of mice treated with scopolamine provided by the present disclosure (left) and distribution of sympathetic nerves of the lacrimal gland 7 days after treated with scopolamine (right);

FIG. 5 shows expressions of adrenergic receptors in lacrimal gland tissues of mice provided by the present disclosure;

FIG. 6 shows results of treatment of dry eye with oral gavage of α1a-, α1d-, α1-, and β-adrenergic receptor inhibitors provided by the present disclosure;

FIG. 7 shows corneal staining by fluorescein sodium (above) and expression levels of inflammatory factors of the lacrimal gland (below) after treatment of dry eye with oral gavage of an α1-adrenergic receptor inhibitor provided by the present disclosure;

FIG. 8 shows tear secretion after treatment of dry eye with intraperitoneal injection of an α1-adrenergic receptor inhibitor provided by the present disclosure;

FIG. 9 shows results after treatment of dry eye with intraperitoneal injection of a medicine for chemical sympathectomy 6-hydroxydopamine (6-OHDA) provided by the present disclosure;

FIG. 10 shows corneal staining by fluorescein sodium (above) and expression levels of inflammatory factors of the lacrimal gland (below) after intraperitoneal injection of 6-hydroxydopamine in dry eye model mice provided by the present disclosure;

FIG. 11 shows tear secretion (left) and ocular surface inflammation (right) in diabetic mice provided by example 7 of the present disclosure;

FIG. 12 shows a change of a norepinephrine level in the lacrimal gland tissues of mice with diabetes provided by the present disclosure;

FIG. 13 shows results of tear secretion after treatment of diabetes-associated dry eye with oral gavage of α1a-, α1-, and β-adrenergic receptor inhibitors provided by the present disclosure;

FIG. 14 shows results of tear secretion after treatment of diabetes-associated dry eye with intraperitoneal injection of an α1-adrenergic receptor inhibitor provided by the present disclosure;

FIG. 15 shows results of tear secretion after treatment of diabetes-associated dry eye with a medicine for chemical sympathectomy 6-hydroxydopamine provided by the present disclosure.

### DETAILED DESCRIPTION OF THE EMBODIMENTS

The present disclosure provides use of a sympathetic activation inhibitor and/or an α1-adrenergic receptor inhibitor in preparing a medicine for treating or relieving dry eye syndrome. The α1-adrenergic receptor inhibitor is a class of medicines capable of selectively binding to an α1-adrenergic receptor, thereby block the combination of a neurotransmitter and catecholamine with the α1a receptor.; and the α1-adrenergic receptor inhibitor has an effect on inhibiting α1a, α1b, and α1d receptors at the same time. The present disclosure uses a tissue clearing method for the first time to find that the lacrimal gland is distributed with abundant sympathetic nerves, indicating that the lacrimal gland has a physiological basis for sympathetic nerves regulation. The present disclosure points out for the first time that the occurrence of dry eye is related to overactivation of sympathetic nerves, inhibiting sympathetic nerve activation or blocking binding of NE to an α1(a) adrenergic receptor can increase tear secretion and relieve dry eye. The sympathetic activation inhibitor and/or the α1-adrenergic receptor inhibitor can be used for treating dry eyes. The α1-adrenergic receptor inhibitor (including an α1a-adrenergic receptor) and the sympathetic activation inhibitor (including a medicine for chemical sympathectomy, a norepinephrine inhibitor and a sympathetic ganglion blocking medicine) are small molecule compounds and have high purity, good stability, low price, and good clinical application prospect. Commercial oral medicines of the α1a-receptor inhibitor include tamsulosin and silodosin, commercial oral medicines of the α1-receptor inhibitor include prazosin, terazosin, doxazosin, alfuzosin, and tramazosin, which are all routine medicines for clinically treating hypertension and prostatic hyperplasia. The norepinephrine inhibitor includes bretylium, reserpine, and guanethidine. The sympathetic ganglion blocking medicine includes trimetaphan camsilate and mecamylamine, which are both medicines for clinically treating hypertension, have a low price, are easy to obtain, and have an application prospect as a new medicine. In the present disclosure, preferably, the dry eye syndrome may include aqueous-deficient dry eye syndrome or diabetes-associated dry eye syndrome.

The present disclosure provides use of a sympathetic activation inhibitor and/or an α1-adrenergic receptor inhibitor in preparing a medicine for increasing tear secretion and/or reduce inflammation of a lacrimal gland and an ocular surface. The medicine prepared by the use of the present disclosure can reverse decrease of tear secretion level caused by sympathetic activation. The specific examples of the present disclosure use a subcutaneous injection of scopolamine to establish a classic model of mice with dry eye and also selects a model of mice with diabetes accompanied by a dry eye complication for experiments. The test results show that compared with normal mice, scopolamine-treated mice and mice with diabetes all show decreased tear secretion and aggravated ocular surface inflammation; at the same time, levels of norepinephrine in plasma and lacrimal gland tissues of the modeled mice are significantly increased, indicating that the occurrence of dry eye is accompanied with overactivation of sympathetic nerves, and inhibition of sympathetic activation by 6-hydroxydopamine (6-OHDA) can significantly increase tear secretion in the two modeled mice. The sympathetic nerves act mainly by releasing norepinephrine to bind to an adrenergic receptor on a target cell. It is found by a quantitative PCR detection that the lacrimal gland cells mainly express four receptors, α1a, α1d, β1, and β2 and an expression level of ala is the highest. The present disclosure takes an α1a adrenergic receptor as a target, uses α1a- and α1-adrenergic receptor inhibitors, and can reverse decreased tear secretion of scopolamine-treated mice and mice with diabetes through intraperitoneal injection and oral gavage, and relieve dry eye symptoms. The present disclosure targets sympathetic nerves, uses a sympathetic activation inhibitor to inhibit sympathetic activation and release of norepinephrine by intraperitoneal injection, and can reverse decreased tear secretion and ocular surface inflammation of scopolamine-treated mice and mice with diabetes, and relieve dry eye symptoms.

In the present disclosure, preferably, the α1-adrenergic receptor inhibitor may include one or more of prazosin, terazosin, doxazosin, alfuzosin, and trimazosin. In the present disclosure, preferably, the α1-adrenergic receptor inhibitor may include an α1a-adrenergic receptor inhibitor. In the present disclosure, preferably, the al a-adrenergic receptor inhibitor may include tamsulosin and/or silodosin. In the present disclosure, preferably, the sympathetic activation inhibitor may include a medicine for chemical sympathectomy, a norepinephrine inhibitor or a sympathetic ganglion blocking medicine; the medicine for chemical sympathectomy may include 6-hydroxydopamine; the norepinephrine inhibitor may include one or more of bretylium, reserpine, and guanethidine; and the sympathetic ganglion blocking medicine may include trimetaphan camsilate and/or mecamylamine.

In the present disclosure, a method for inhibiting sympathetic activation (i.e., an intervention method targeting sympathetic nerves) can also relieve or treat dry eye symptoms. Preferably, the method for inhibiting sympathetic activation may include an acupuncture for inhibiting sympathetic activation or a sympathetic nerve block. Sources of the above specific medicines are not particularly limited in the present disclosure, as long as conventional commercially available products of the medicines well known to those skilled in the art may be used.

In the present disclosure, preferably, the α1-adrenergic receptor inhibitor or an agent inhibiting sympathetic activation may have a concentration of 1-100 mg/kg in the medicine. In the present disclosure, preferably, a dosage of the medicine may include an oral medicine, an injection, a sustained release tablet, a nasal spray or an embedded sustained release tablet. In the present disclosure, the embedded sustained release tablet may preferably be administered locally near the lacrimal gland (e.g., at the conjunctiva). Preparation methods of the nasal spray or the embedded sustained release tablet are not specifically limited in the present disclosure, as long as the preparation methods of the nasal spray or the embedded sustained release tablet well known in the art may be used.

In the present disclosure, preferably, alfuzosin may be prepared into a nasal spray or a sustained-release tablet; and 6-hydroxydopamine may be prepared into an injection. In the present disclosure, preferably, the injection may be prepared by dissolving 100 mg/kg of 6-hydroxydopamine (6-OHDA) in 0.02% VC (solvent is 0.9% NaCl aqueous solution) and ready-to-use and ready-made.

The use of a sympathetic activation inhibitor and/or an α1-adrenergic receptor inhibitor in preparing a medicine for treating dry eye symptoms provided by the present disclosure will be further described in detail below in conjunction with examples, and the technical solutions of the present disclosure include but are not limited to the following examples.

### Example 1

Decreased Tear Secretion and Aggravated Ocular Surface Inflammation in Model of Mice Treated with Scopolamine

Female C57BL/6 mice were selected for the experiment and placed in a perforated cage with a constant fan flow and a humidity <20%. The modeled mice with dry eye were induced by subcutaneous injection of scopolamine (0.5 mg/0.2 ml, 3 times a day) for 7 consecutive days (refer to the following literature: Wu Y, Bu J, Yang Y, et al. Therapeutic effect of MK2 inhibitor on experimental murine dry eye. Invest Ophthalmol Vis Sci. 2017; 58:4898-4907), the mice not treated and fed under a normal condition were used as a control, and the experimental mice had no corneal defects and neovascularization or conjunctival damage.

### 1) Tear Secretion

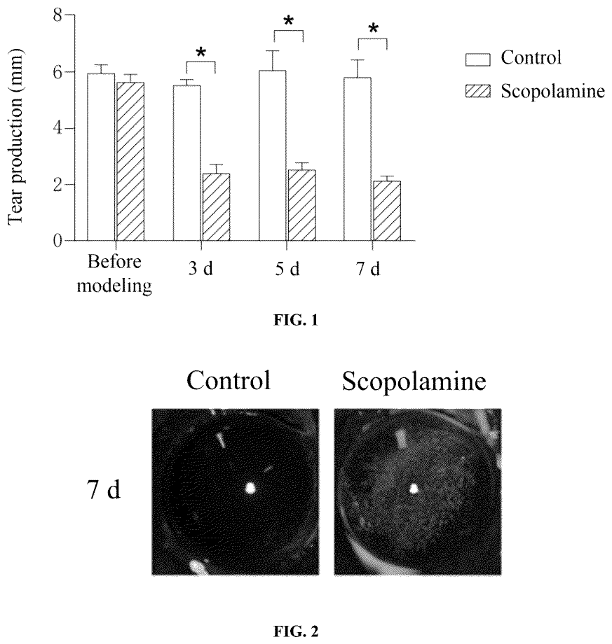

A phenol red cotton thread was used to detect the tear production of mice. One end of the phenol red cotton thread was placed at an inner ⅓ of the conjunctival sac of the lower eyelid, after 20 s, the phenol red cotton thread was taken out, and the length of the cotton thread wetted with tears was measured. The result was shown in FIG. 1 (tear secretion after modeling of mice treated with scopolamine). Compared with the mice in the control group, the mice in the scopolamine group had significantly reduced tear secretion after 3 days of modeling, which was maintained until the 7th day of the observation period.

### 2) Corneal Staining by Fluorescein Sodium and Scoring

Staining by fluorescein sodium was used to observe damage of a corneal epithelial barrier function, observation was performed with a slit lamp, and photographing was performed. The result was shown in FIG. 2 (corneal staining by fluorescein sodium 7 days after modeling of mice treated with scopolamine). Compared with the mice in the control group, the mice in the scopolamine group were positive in staining by fluorescein sodium in scopolamine 7 days after modeling.

### 3) Inflammation of Lacrimal Gland and Cornea

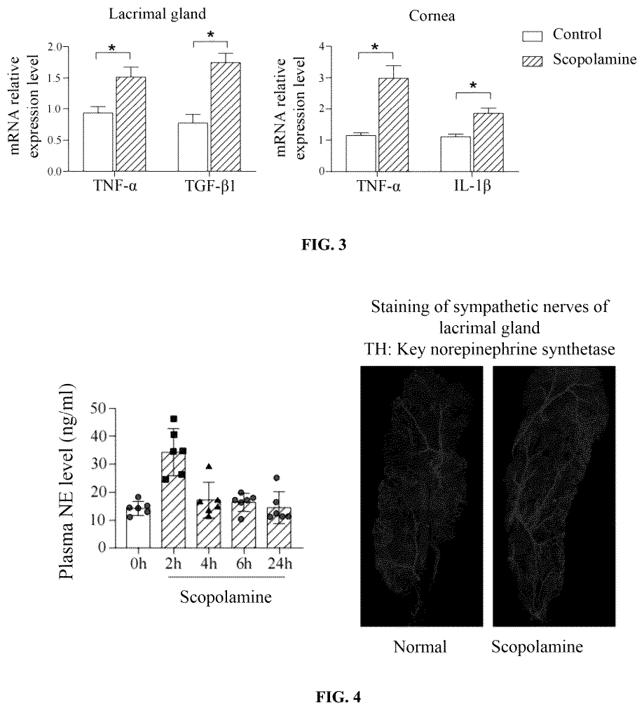

After 7 days of modeling, the lacrimal glands and the corneas of the mice were taken to extract mRNA. Expression levels of inflammatory factors TNFα, TGFβ1, and IL-1β were detected by qPCR. The result was shown in FIG. 3 (expression levels of inflammatory factors of the lacrimal glands (left) and the corneas (right) 7 days after modeling of mice treated with scopolamine). Compared with the mice in the control group, the mice in the scopolamine group had up-regulated expressions of inflammatory factors of TNFα and TGFβ1 in the lacrimal glands and up-regulated expressions of TNFα and IL-1β in the corneas.

### Example 2

Overactivation of Sympathetic Nerves in Model of Mice Treated with Scopolamine

A model of mice treated with scopolamine was established according to the method in example 1. The mouse plasma was collected at 0 h (that is, mice as a normal control), 2 h, 4 h, 6 h, and 24 h after injection of scopolamine, a level of norepinephrine (NE) was detected by a norepinephrine enzyme-linked immunosorbent assay kit (purchased from Abnova, Art. No.: ABN-KA1891). The result was shown in FIG. 4 (left). The level of the norepinephrine in the blood of the modeled mice was significantly increased 2 h after the injection of the scopolamine and reached a peak, and returned to a normal level 4 h after the injection. The result indicated overactivation of sympathetic nerves in a model of mice treated with scopolamine.

A modified CE-3D clearing staining method was used, the lacrimal gland was fixed with 4% PFA at a room temperature for 2 h, washed with a PBS buffer, and permeabilized and blocked overnight, the blocked lacrimal gland was incubated with a primary antibody (sympathetic nerve TH) for 2 days, washed, and incubated with a secondary antibody (Alexa-647) for 1 day, the lacrimal gland was washed overnight with triton+0.5% 1-thioglycerol, soaked in a clearing solution for 2 days, and photographed by a confocal microscope. The result was shown in FIG. 4 (right). Dense sympathetic nerves were distributed in the lacrimal glands of the normal mice. The thick and big sympathetic nerves in the lacrimal glands of the mice treated with scopolamine were increased and density of nerve fibers was increased, which proved that the sympathetic nerves of the mice treated with scopolamine were overactivated.

FIG. 4 showed a change of a norepinephrine (NE) level in the blood of the mice treated with scopolamine (left) and distribution of sympathetic nerves of the lacrimal gland 7 days after modeling of the mice treated with scopolamine (right).

### Example 3

### Identification of Target Receptors for Sympathetic Nerves to Regulate Secretion of Lacrimal Gland

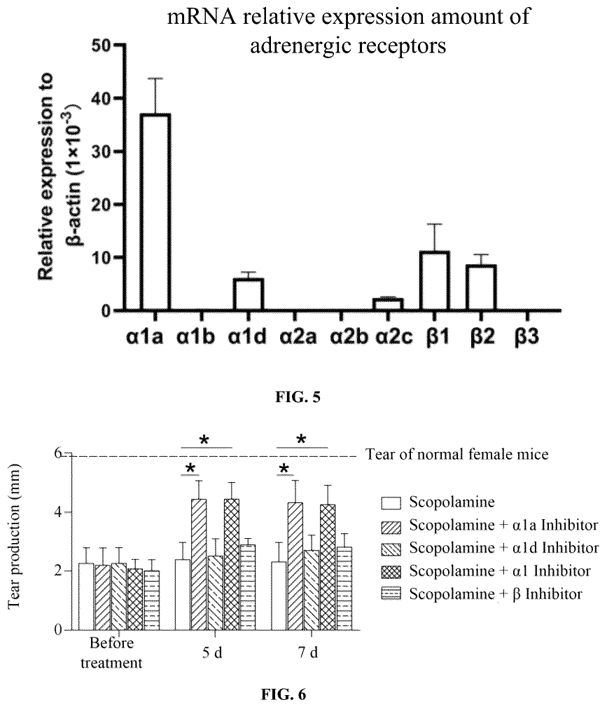

Sympathetic nerves act mainly by releasing norepinephrine (NE) to bind to an adrenergic receptor on a target cell. mRNA was extracted from a normal lacrimal gland tissue of mice and expressions of adrenergic receptors in lacrimal gland cells were detected by quantitative PCR. The result was shown in FIG. 5 (expressions of adrenergic receptors in lacrimal gland tissues of mice). The lacrimal gland cells mainly expressed four receptors, α1a, α1d, β1, and β2, and an expression level of α1a was the highest, indicating that the α1a receptor of the lacrimal gland cells may be a main target receptor for sympathetic nerves to regulate tear secretion.

### Example 4

### Therapeutic Effect of Oral Administration of α1a- and α1-Adrenergic Receptor Inhibitors on Dry Eye

A model of mice treated with scopolamine was established according to the method of example 1.

The mice in the scopolamine experimental group were gavaged with 1-10 mg/kg of α1a-, α1d-, α1-, and β-adrenergic receptor inhibitors (200 μl/mice) once a day on the 3rd day after modeling. The mice in a control group (compared to scopolamine) were gavaged with normal saline. The tear secretion of the mice was observed 5 days and 7 days after the treatment. The result was shown in FIG. 6 (tear secretion after treatment of dry eye with oral gavage of α1a-, α1d-, α1-, and β-adrenergic receptor inhibitors). Commercial oral medicines of the α1a- and the α1-adrenergic receptor inhibitors can improve the tear secretion in the mice treated with scopolamine, while the α1d- and the β-receptor inhibitors had no significant intervention effect.

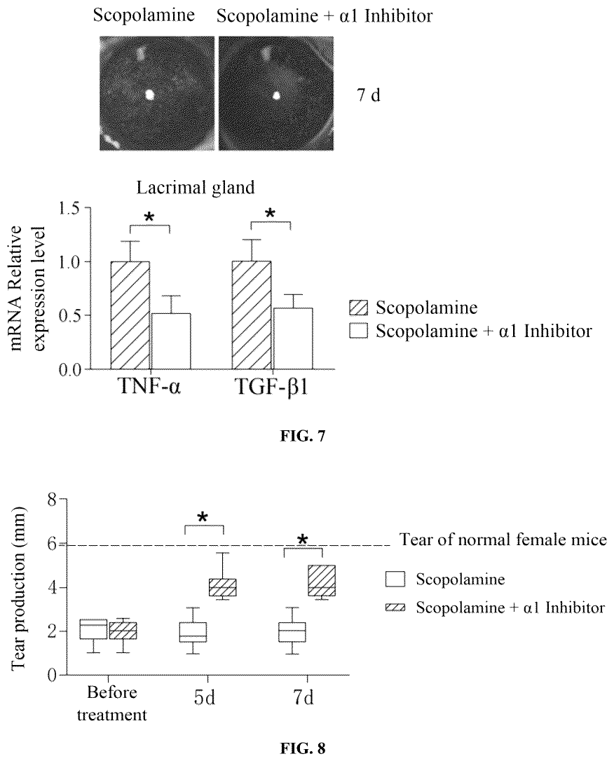

After 7 days of the treatment, staining by sodium fluorescein was used to observe damage of a corneal epithelial barrier function. The lacrimal glands of the mice were taken for qPCR detection. The result was shown in FIG. 7 (corneal staining by fluorescein sodium and expression levels of inflammatory factors of the lacrimal gland of treatment of dry eye with oral gavage of an α1-adrenergic receptor inhibitor, and corneal staining by fluorescein sodium (above) and expression levels of inflammatory factors of the lacrimal gland (below)), the α1-adrenergic receptor inhibitor can reduce staining of an ocular surface by fluorescein sodium and decrease expression levels of inflammatory factors TNFα and TGFβ1 of the lacrimal gland.

### Example 5

### Therapeutic Effect of Intraperitoneal Injection of α1-Adrenergic Receptor Inhibitor on Dry Eye

A model of mice treated with scopolamine was established according to the method of example 1.

The mice in the scopolamine experimental group were intraperitoneally injected with 1-10 mg/kg of an α1-adrenergic receptor inhibitor (200 μl/mice) once a day on the 3rd day after modeling. The mice in a control group (compared to scopolamine) were intraperitoneally injected with normal saline. The tear secretion of the mice was observed 5 days and 7 days after the treatment. The result was shown in FIG. 8 (treatment of dry eye with intraperitoneal injection of an α1-adrenergic receptor inhibitor). The α1-adrenergic receptor inhibitor can significantly improve the tear secretion in the mice treated with scopolamine.

### Example 6

### Relieving Effect of Chemical Sympathectomy on Dry Eye

A model of mice treated with scopolamine was established according to the method of example 1.

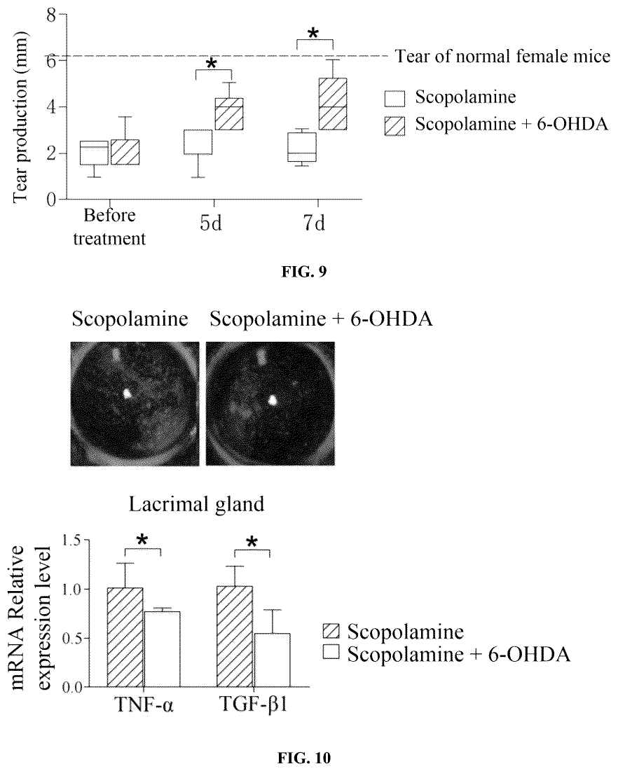

The mice in the scopolamine experimental group were intraperitoneally injected with (100 mg/kg) of a medicine for chemical sympathectomy 6-hydroxydopamine once a day on the 3rd day after modeling consecutively for 4 days. The mice in a control group (compared to scopolamine) were intraperitoneally injected with normal saline. The tear secretion of the mice was observed 5 days and 7 days after the treatment. The result was shown in FIG. 9 (results after treatment of dry eye with intraperitoneal injection of a medicine for chemical sympathectomy 6-hydroxydopamine (6-OHDA)). The medicine for chemical sympathectomy 6-hydroxydopamine can significantly increase the tear secretion in the mice treated with scopolamine.

On the 7th day of the treatment, staining by sodium fluorescein was used to observe damage of a corneal epithelial barrier function. The lacrimal glands of the mice were taken for qPCR detection. The result was shown in FIG. 10 (corneal staining by fluorescein sodium and expression levels of inflammatory factors of the lacrimal gland of treatment of dry eye with intraperitoneal injection of 6-hydroxydopamine, and corneal staining by fluorescein sodium (above) and expression levels of inflammatory factors of the lacrimal gland (below)), the 6-hydroxydopamine can reduce staining of an ocular surface by fluorescein sodium and decrease expression levels of inflammatory factors TNFα and TGFβ1 of the lacrimal gland.

### Example 7

Decreased Tear Secretion and Aggravated Ocular Surface Inflammation in Model of Mice with Diabetes

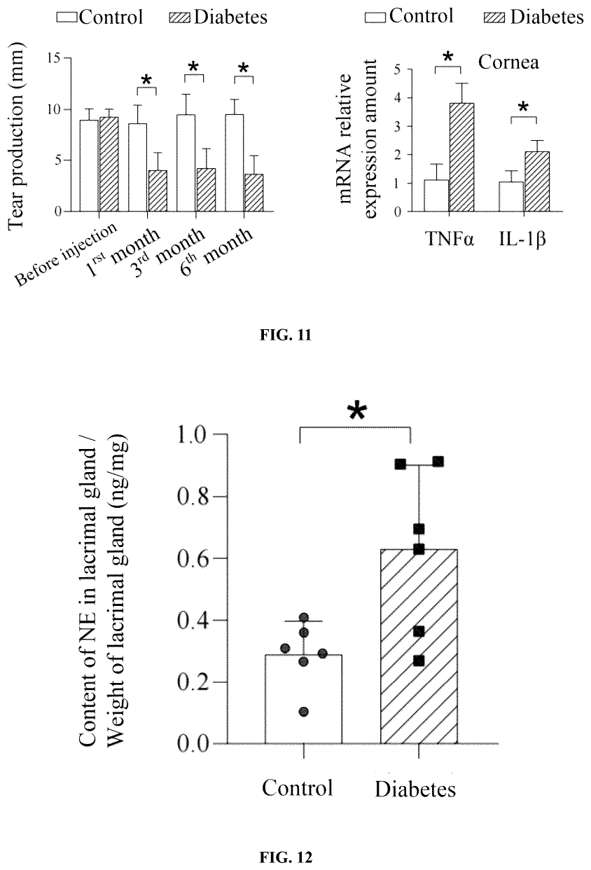

Male C57BL/6 mice were selected, a type 1 diabetes model was established by intraperitoneal injection of streptozotocin (STZ) (the establishment method refers to the prior art: Lingling, Yang, Guohu, Di, Xia, & Qi, et al. Substance p promotes diabetic corneal epithelial wound healing through molecular mechanisms mediated via the neurokinin-1 receptor. Diabetes. 2014; 63: 4262-4274.), and the mice with blood glucose value greater than 300 mg/dL were screened for experiments after 1 month. The mice not treated and fed under a normal condition were used as a control, and the experimental mice had no corneal defects and neovascularization or conjunctival damage. The result was shown in FIG. 11 (tear secretion and ocular surface inflammation after modeling of mice with diabetes, tear secretion (left) and ocular surface inflammation (right)). Compared with the mice in the control group of the same age, the mice with diabetes showed significantly decreased tear secretion 1 month after STZ injection, which was maintained until the 6th month of the observation period; and compared with the mice in the control group of the same age, the mice with diabetes had up-regulated inflammatory factors TNFα and IL-1β in the corneas, indicating that the mice with diabetes exhibited main features of dry eye, such as decreased tear secretion and ocular surface inflammation.

### Example 8

Overactivation of Sympathetic Nerves Mice with Diabetes

A model of mice with diabetes was established according to the method of example 7.

The lacrimal glands of the mice were removed. A level of norepinephrine (NE) was detected by a norepinephrine enzyme-linked immunosorbent assay kit (purchased from Abnova, Art. No.: ABN-KA3836). The result was shown in FIG. 12 (a change of a norepinephrine level in the lacrimal gland tissues of mice with diabetes).

Compared with the mice in the normal control group, the mice with diabetes had an increased level of NE in the lacrimal glands, indicating an overactivation of sympathetic nerves in the lacrimal glands of the mice with diabetes.

### Example 9

### Therapeutic Effect of Oral Administration of α1a- and α1-Adrenergic Receptor Inhibitors on Diabetes-Associated Dry Eye

A model of mice with diabetes was established according to the method of example 7.

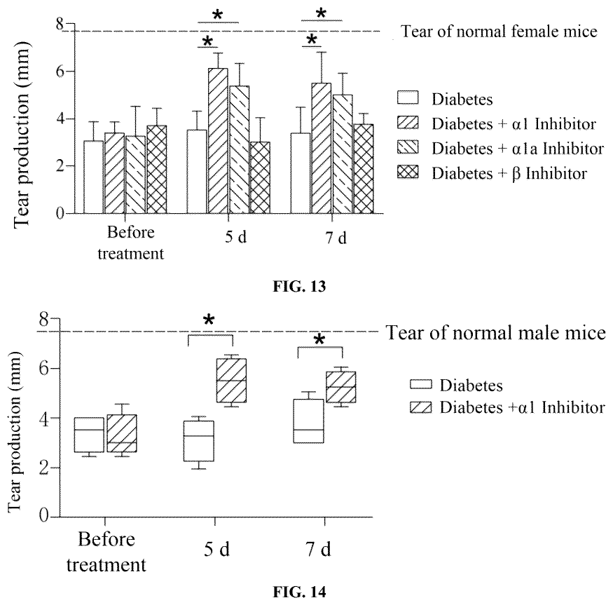

The mice in the diabetes experimental group were gavaged with 1-10 mg/kg of commercial oral medicines of α1a-, α1-, and β-adrenergic receptor inhibitors (200 μl/mice) once a day. The mice in a control group (compared to diabetes) were gavaged with normal saline. The tear secretion of the mice was observed 5 days and 7 days after the treatment. The result was shown in FIG. 13 (tear secretion after treatment of diabetes-associated dry eye with oral gavage of α1a- and α1-adrenergic receptor inhibitors). Commercial oral medicines of the α1a- and the α1-adrenergic receptor inhibitors can improve the tear secretion in the mice with diabetes, while the β-receptor inhibitor had no significant intervention effect.

### Example 10

### Therapeutic Effect of Intraperitoneal Injection of α1-Adrenergic Receptor Inhibitor on Diabetes-Associated Dry Eye

A model of mice with diabetes was established according to the method of example 7.

The mice in the diabetes experimental group were intraperitoneally injected with 1-10 mg/kg of an α1-adrenergic receptor inhibitor (200 μl/mice) once a day. The mice in a control group (compared to diabetes) were intraperitoneally injected with normal saline. The tear secretion of the mice was observed 5 days and 7 days after the treatment. The result was shown in FIG. 14 (tear secretion after treatment of diabetes-associated dry eye with intraperitoneal injection of an α1-adrenergic receptor inhibitor). The α1-adrenergic receptor inhibitor can improve the tear secretion in the mice with diabetes.

### Example 11

### Therapeutic Effect of Chemical Sympathectomy on Diabetes-Associated Dry Eye

A model of mice with diabetes was established according to the method of example 7.

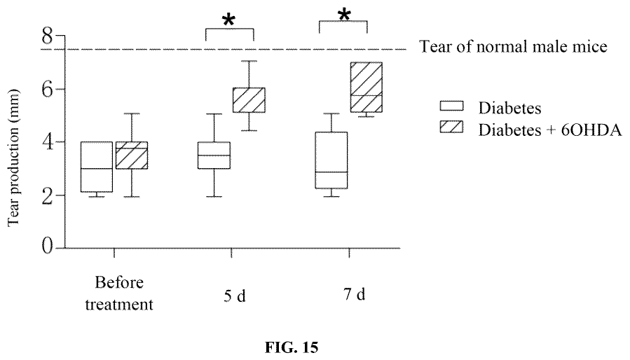

The mice in the diabetes experimental group were intraperitoneally injected with (100 mg/kg) of a medicine for chemical sympathectomy 6-hydroxydopamine once a day consecutively for 4 days. The mice in a control group (compared to diabetes) were intraperitoneally injected with normal saline. The tear secretion of the mice was observed 5 days and 7 days after the treatment. The result was shown in FIG. 15 (results of tear secretion after treatment of diabetes-associated dry eye with a medicine for chemical sympathectomy 6-hydroxydopamine). The medicine for chemical sympathectomy 6-hydroxydopamine can significantly increase the tear secretion in the mice with diabetes.

It can be seen from the results of the above examples that the present disclosure can improve tear secretion and relieve dry eye symptoms in mice by inhibiting sympathetic activation or antagonizing α1a- and α1-adrenergic receptors by a medicine. At the same time, the α1a- and α1-adrenergic receptor inhibitors, the sympathetic activation inhibitor, and a sympathetic ganglion blocking medicine are small molecule compounds, and have high purity, good stability, low price, and good clinical application prospect.

Commercial oral medicines of the α1a-receptor inhibitor include tamsulosin and silodosin. The commercial oral medicines of the α1-receptor inhibitor include prazosin, terazosin, doxazosin, alfuzosin, and tramazosin, which are all routine medicines for clinically treating hypertension and prostatic hyperplasia, have a low price, are easy to obtain, and have an application prospect as a new medicine.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1.-9. (canceled)

10. Use of a sympathetic activation inhibitor and/or an α1-adrenergic receptor inhibitor in preparing a medicine for treating or relieving dry eye syndrome.

11. The use according to claim 10, wherein the dry eye syndrome comprises aqueous-deficient dry eye syndrome or diabetes-associated dry eye syndrome.

12. The use according to claim 11, wherein the α1-adrenergic receptor inhibitor comprises one or more of prazosin, terazosin, doxazosin, alfuzosin, and trimazosin.

13. The use according to claim 11, wherein the α1-adrenergic receptor inhibitor comprises an α1a-adrenergic receptor inhibitor.

14. The use according to claim 13, wherein the α1a-adrenergic receptor inhibitor comprises tamsulosin and/or silodosin.

15. The use according to claim 11, wherein the sympathetic activation inhibitor comprises a medicine for chemical sympathectomy, a norepinephrine inhibitor or a sympathetic ganglion blocking medicine; the medicine for chemical sympathectomy comprises 6-hydroxydopamine; the norepinephrine inhibitor comprises one or more of bretylium, reserpine, and guanethidine; and the sympathetic ganglion blocking medicine comprises trimetaphan camsilate and/or mecamylamine.

16. The use according to claim 11, wherein the α1-adrenergic receptor inhibitor or an agent inhibiting sympathetic activation has a concentration of 1-100 mg/kg in the medicine.

17. The use according to claim 11, wherein a dosage of the medicine comprises an oral medicine, an injection, a sustained release tablet, a nasal spray or an embedded sustained release tablet.

19. The use according to claim 10, wherein the α1-adrenergic receptor inhibitor comprises one or more of prazosin, terazosin, doxazosin, alfuzosin, and trimazosin.

19. The use according to claim 10, wherein the α1-adrenergic receptor inhibitor comprises an α1a-adrenergic receptor inhibitor.

20. The use according to claim 19, wherein the α1a-adrenergic receptor inhibitor comprises tamsulosin and/or silodosin.

21. The use according to claim 10, wherein the sympathetic activation inhibitor comprises a medicine for chemical sympathectomy, a norepinephrine inhibitor or a sympathetic ganglion blocking medicine; the medicine for chemical sympathectomy comprises 6-hydroxydopamine; the norepinephrine inhibitor comprises one or more of bretylium, reserpine, and guanethidine; and the sympathetic ganglion blocking medicine comprises trimetaphan camsilate and/or mecamylamine.

22. The use according to claim 10, wherein the α1-adrenergic receptor inhibitor or an agent inhibiting sympathetic activation has a concentration of 1-100 mg/kg in the medicine.

23. The use according to claim 10, wherein a dosage of the medicine comprises an oral medicine, an injection, a sustained release tablet, a nasal spray or an embedded sustained release tablet.

24. Use of a sympathetic activation inhibitor and/or an α1-adrenergic receptor inhibitor in preparing a medicine for increasing tear secretion and/or reduce inflammation of a lacrimal gland and an ocular surface.

25. The use according to claim 24, wherein the α1-adrenergic receptor inhibitor comprises one or more of prazosin, terazosin, doxazosin, alfuzosin, and trimazosin.

26. The use according to claim 24, wherein the α1-adrenergic receptor inhibitor comprises an α1a-adrenergic receptor inhibitor.

27. The use according to claim 24, wherein the α1a-adrenergic receptor inhibitor comprises tamsulosin and/or silodosin.

28. The use according to claim 24, wherein the sympathetic activation inhibitor comprises a medicine for chemical sympathectomy, a norepinephrine inhibitor or a sympathetic ganglion blocking medicine; the medicine for chemical sympathectomy comprises 6-hydroxydopamine; the norepinephrine inhibitor comprises one or more of bretylium, reserpine, and guanethidine; and the sympathetic ganglion blocking medicine comprises trimetaphan camsilate and/or mecamylamine.

29. The use according to claim 24, wherein the α1-adrenergic receptor inhibitor or an agent inhibiting sympathetic activation has a concentration of 1-100 mg/kg in the medicine.
